(19) United States
(12) Patent Application Publication
(10) Pub. No.: US 2009/0291490 A1
(43) Pub. Date: Nov. 26, 2009

(54) Photo-Bioreactor

(75) Inventor: Drew M. Spradling, Wheeling, WV (US)

Correspondence Address: PHILIP D. LANE, P.O. BOX 79318, CHARLOTTE, NC, 28271-7063, US

(73) Assignee: Touchstone Research Laboratory, Ltd.

(21) Appl. No.: 12/355,792

(22) Filed: Jan. 18, 2009

Related U.S. Application Data
(60) Provisional application No. 61/021,966, filed on Jan. 18, 2008.

(51) Int. Cl. C12M 1/00

(52) U.S. Cl. 435/292.1

(57) ABSTRACT

A photo-bioreactor may be constructed from multiple layers of polymer film arranged to provide a cultivation region and an energy modulation region. The energy modulation region may include a phase change material. A plurality of photo-bioreactors may be positioned in series with one another and arranged in a variety of configurations.

### CROSS-REFERENCE TO RELATED APPLICATIONS

This application claims priority to U.S. Provisional Patent Application No. 61/021,966, filed Jan. 18, 2008, which is herein specifically incorporated by reference in its entirety.

### BRIEF SUMMARY OF THE INVENTION

A photo-bioreactor may be constructed from multiple layers of polymer film arranged to provide a cultivation region and an energy modulation region. In some embodiments one or more insulation region may be utilized. The photo-bioreactor may have a plurality of ports for introducing and removing material from the cultivation region. Further, a plurality of photo-bioreactors may be positioned in series with one another and arranged in a variety of configurations.

In some embodiment, photo-bioreactor may comprise a cultivation region defined between two opposing layers of polymer film, wherein at least one of two opposing layers of polymer film absorbs less that about 15% of the natural sunlight. An energy modulation region may be positioned adjacent to the cultivation region, wherein the energy modulation region comprises an energy modulating material comprising a phase change material. In some embodiment, the phase change material may comprise a parrafin wax. In some embodiments, the paraffin wax may have the general formula CₙH₂ₙ₊₂, where n may range from about 12 to about 20. In further embodiment, the phase change material may include tetradecane, hexadecane, octadecane, eicosane, Kenwax 18, Kenwax 19, or 60% neopentyl glycol/40% pentaglycerine, or combinations thereof. Still further, the phase change material may comprise at least two different phase change materials. In additional embodiments, the photo-bioreactor may further include an insulation region positioned adjacent to the side of the cultivation region opposite the energy modulation region. Still further, the photo-bioreactor may include 2 or more insulation regions.

### BRIEF DESCRIPTION OF THE FIGURES

FIG. 1 is a cross-sectional representation a photo-bioreactor in accordance with an embodiment of the invention.

FIG. 2 illustrates a photo-bioreactor in accordance with an embodiment of the invention.

FIG. 3 illustrates a configuration of the cultivation region in accordance with an embodiment of the invention.

FIG. 4 illustrate another configuration of the cultivation region in accordance with an embodiment of the invention.

FIG. 5 illustrate another configuration of the cultivation region in accordance with an embodiment of the invention.

FIG. 6 illustrate another configuration of the cultivation region in accordance with an embodiment of the invention.

FIG. 7 illustrates a plurality of photo-bioreactors on a support frame in accordance with an embodiment of the invention.

FIG. 8 illustrates a plurality of photo-bioreactors extending vertically on a support frame in accordance with an embodiment of the invention.

### DETAILED DESCRIPTION OF EMBODIMENTS OF THE INVENTION

A photo-bioreactor system for growing and cultivating small organisms, such as algae, in a solvent system, such as water, is described. The photo-bioreactor system may be used with a variety of organisms, including, but not limited to, algae, microalgae, plankton, bacteria, and other similar plants and organisms. Generally, the photo-bioreactor is a closed system that is not open to the ambient atmosphere.

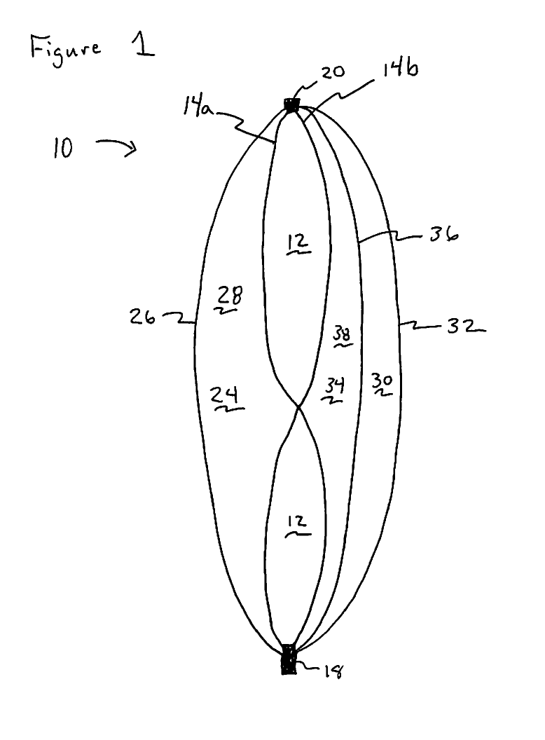

With reference to FIG. 1, there is illustrated a cross-sectional representation of a photo-bioreactor 10 in accordance with an embodiment of the invention. The photo-bioreactor includes a cultivation region 12. The cultivation region 12 is defined by opposing layers 14a and 14b of a polymer film. In some embodiments, the polymer film may be any of the green house films commercially available. In some embodiments the polymer film may include, but is not limited to, a polyethylene film and/or a polypropylene film. The polymer film may be any polymer film that absorbs less than about 15% of natural sunlight. In some embodiments, the polymer film may have a thickness ranging from about 2 mil to about 12 mil.

With reference to FIG. 2 and continuing reference to FIG. 1, the opposing polymer film layers 14a and 14b are sealed to each other at various points and/or edges such that the cultivation region 12 is in the form of a continuous channel 16. The film layers 14a and 14b may be sealed by the use of an adhesive, or more preferably, the layers may be sealed to each other by applying sufficient heat to the point on the layers in which the seal is desired. Such heat sealing methods of polymer films are well known to those skilled in the art.

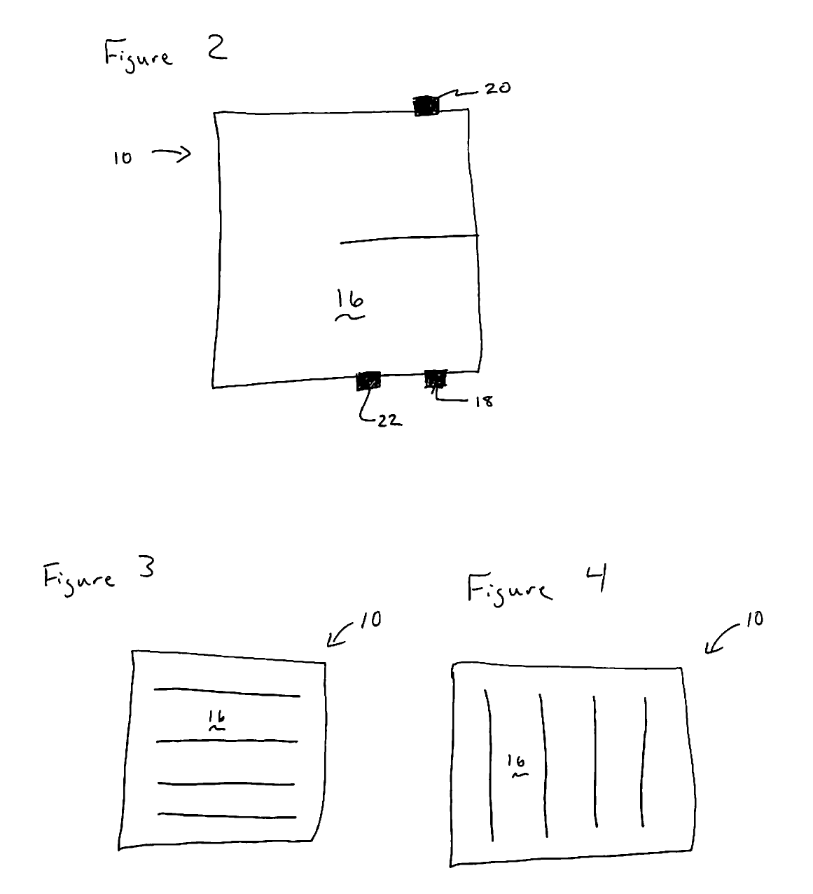

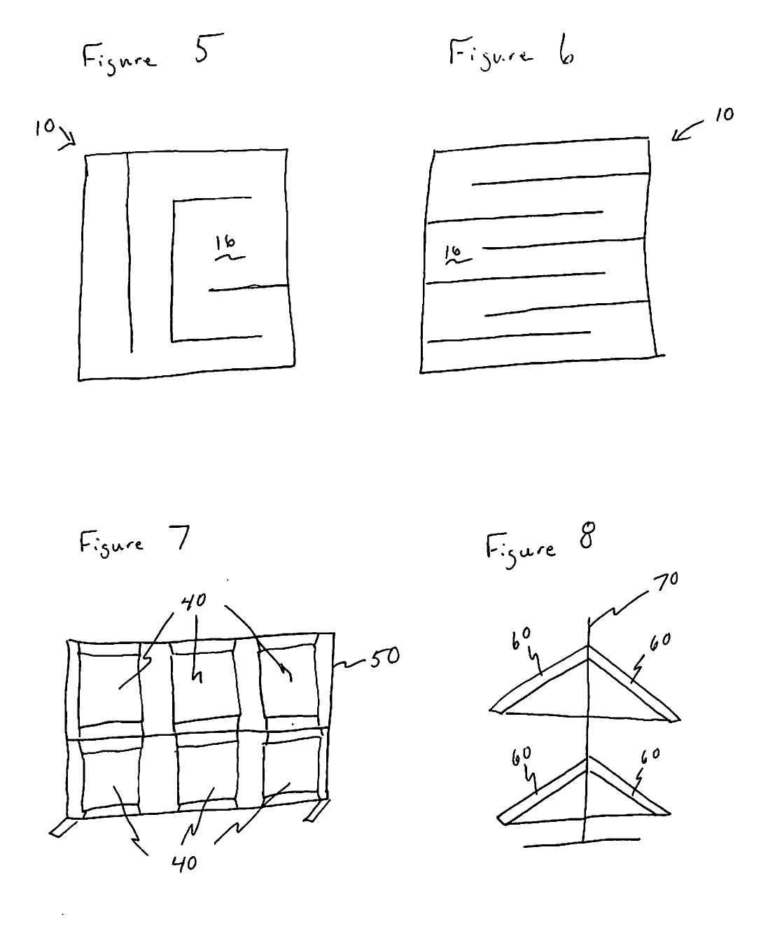

The continuous channel 16 of the cultivation region 12 may be arranged in a variety of configurations. With reference to FIGS. 3, 4, 5, and 6, such configurations may include predominantly horizontal channels as shown in FIG. 3, or predominantly vertical channels as shown in FIG. 4. In some embodiments, the continuous channel 16 may be a combination of generally horizontal and vertical channels as shown in FIG. 5. In additional embodiments, the continuous channel may be a series of horizontal or vertical channels arranged in a serpentine type configuration as illustrated in FIG. 6. The design of the particular continuous channel is not particularly limited and may include a wide range of pathways and designs. Further, more than one continuous channel may be created between the opposing layers 14a and 14b of polymer film.

With reference to FIGS. 1 and 2, the photo-bioreactor 10 may include one or more ports designed to allow fluid, gases, and/or nutrients to enter or exit the cultivation region 12. The ports may be an opening between the layers of polymer film forming the cultivation region. The opening may be fitted with any number of fittings for allowing the connection of pipes or tubing to the photo-bioreactor. The photo-bioreactor 10 may include at least one inlet port 18 for introducing fluid into the reactor. The location of the inlet port 18 is not particularly limited, but may generally be placed near one end of the continuous channel 16. The number of inlet ports is not particularly limited, and may range from about 1 to about 10 or more. As the size of the photo-bioreactor increases, it may be desirable to include more inlet ports. Further, if more than one continuous channel is utilized, an inlet port for each continuous channel may be desirable.

An exit port 20 may be included in the photo-bioreactor 10. The exit port allows for fluid and/or gases to exit the photo-bioreactor. The exit port may be positioned at virtually any location along the continuous channel 16. In certain embodiments, the exit port 20 is located near the opposite end of the continuous channel in which the inlet port 18 is located. The exit port 20 may be configured in the same way as the inlet port 18, described above. If desired a single port may be utilized as both an inlet port and an exit port at different times of operation.

Optionally, the photo-bioreactor may be fitted with one or more additional ports, such as additional port 22. The additional port 22 may be used to introduce additional materials to the photo-bioreactor, such as gases or nutrients. Further, additional ports may be used to allow various instruments to enter the cultivation region 12. Such instrument may include thermocouples, oxygen sensors, carbon dioxide sensors, and other similar instruments for monitoring the condition in the cultivation region 12.

With most photo-bioreactors systems, it is desirable to control the temperature in the cultivation region 12 to promote or control the growth of the organism. Typically, the photo-bioreactor may be placed in a climate controlled room or in a greenhouse. With the present invention, an insulation region 24 may be positioned on one or more sides of the cultivation region 12 to aid in moderating the temperature in the cultivation region 12. In certain embodiments, the insulation region 24 may be created by applying a layer of polymer film 26 over polymer film layer 14a with an insulating material 28 positioned between polymer film 26 and polymer film layer 14a. The insulating material 28 may include, but is not limited to air, gas, fiberglass, cotton, fibrous insulating material, and other insulating materials.

If desired, an additional insulation region 30 may be positioned near the cultivation region 12 and generally near the opposite side of the cultivation region as the insulation region 24. The additional insulation region 30 may be configured in a similar manner as for insulation region 24 described above using a layer of polymer film 32. If light is necessary for the growth of the desired organism, and the photo-bioreactor is utilizing insulating regions on opposing sides of the cultivation region, at least one of the insulation regions should be filled with an insulating material that allows for sufficient light to penetrate the insulating layer and reach the cultivating region 12. The amount of light sufficient to grow a particular organism will vary with operating conditions and the particular type of organism.

To assist in the control of the temperature in the cultivation region 12, an energy modulation region 34 may be positioned near the cultivation region 12. The energy modulation region helps maintain the temperature of the cultivation region within desired temperature ranges or parameters. The energy modulation region 34 may be defined, in some embodiments, by the use of another polymer film 36 affixed to the polymer film layer 14b. In the embodiment illustrated in FIG. 1, the energy modulation region 34 may be positioned between the cultivation region 12 and the additional insulation region 30. The energy modulating region may contain an energy modulation material 38 that absorbs heat/energy as the ambient temperature increases, and releases energy when the ambient temperature decreases.

The energy modulating material may include a phase change material. A phase change material is a material that absorbs heat/energy when the temperature is over a specified temperature and releases heat/energy by going through a phase transition, such as from liquid to solid, when the temperature drops below a certain temperature. Solid-liquid phase change materials perform like conventional storage materials; their temperature rises as they absorb heat. Unlike conventional storage materials, however, when phase change materials reach the temperature at which they change phase (generally the melting point or softening point), they absorb large amounts of heat without a significant rise in temperature. When the ambient temperature around the liquid material falls, the phase change material solidifies, releasing its stored latent heat. In some embodiments the phase change material has the ability to store more than 10 times more heat per unit volume than conventional storage materials such as water, masonry, or rock. The phase change material needs to have a reversible phase transition, a high latent heat, and preferably small changes in volume between phases and preferably a low vapor pressure. In certain embodiments, the phase change material is transparent to photosynthetically active radiation. In further embodiments, the phase change material exhibits a phase transition temperature ranging from about 40° F. to about 80° F. With a phase transition temperature ranging from about 40° F. to about 80° F., significant heat storage occurs during the day and significant heat is release at night. The incorporation of a phase change material as part of the photo-bioreactor allows for the absorption of latent heat from the solar spectrum and the subsequent release of the stored heat to the cultivation region when the temperature drops.

In some embodiments, the phase change material may include paraffin wax. Paraffin waxes may comprise about 75% alkanes. Paraffin waxes can contain several alkanes, resulting in a melting range rather than a melting point. As the molecular weight increases, the melting point or melting range tends to increase as well. Using different mixtures of alkanes, specific transition temperatures for paraffin waxes may be attained. In further embodiments, phase change material may comprises paraffin wax having the general formula CₙH₂ₙ₊₂ where n may range from about 12 to about 20. In additional embodiments, phase change materials may include, but are not limited to, tetradecane, hexadecane, octadecane, eicosane, Kenwax 18 (Outlast Technology), Kenwax 19 (Outlast Technology), 60% neopentyl glycol/40% pentaglycerine, and combination thereof. Blends of phase change materials may be used such that the phase transition occurs at a desired temperature. Various phase change materials may have phase transition temperatures that range from about 40° F. to about 100° F.

While FIG. 1 illustrates the energy modulation region 34 positioned between the cultivation region 12 and the additional insulation region 30. The additional insulation region 30 and/or the insulation region 24 may be optional. Depending upon the ambient conditions, and the temperatures necessary for the growth of the organism, the photo-bioreactor with one or more of the insulation region 24, the energy modulation region 34, and/or the additional insulation region 30 may be utilized to control the temperature in the cultivation region 12 of the photo-bioreactor 10. In cold climates a supplemental heat source may be incorporated as part of the photo-bioreactor. For example, heat tape or heating elements may be integrated into the photo-bioreactor. In other embodiments, a water heater may be used heat the water or culture medium as it circulates through the photo-bioreactor. The supplemental heat source may be activate when the temperature of the water or culture medium reaches a predetermined minimum temperature.

The size of the photo-bioreactor 10 is not particularly limited and may range from a few inches to several feet. The design of the photo-bioreactor should take into account the organism to be cultivated. If the system utilizes a solvent system such as water, the weight of the solvent should be factored in for the design of the photo-bioreactor, with respect to size of the reactor, volume of the cultivation region, types of materials, and other parameters.

As illustrated in FIG. 7, one or more photo-bioreactors 40 may be hung or mounted on a support frame 50. The photo-bioreactors 40 may be positioned vertically, horizontally, or at an angle between vertical and horizontal. In some embodiments, two or more photo-bioreactors may be supported on one or more support frames. The two or more photo-bioreactors may be positioned along side one another in a general vertical, horizontal, or angled configuration, or some combination of orientations. With respect to FIG. 8, in some embodiments, two or more photo-bioreactors 60 may be positioned or stacked vertically over one another on a support frame 70. When positioning two or more photo-bioreactors over one another, in some embodiments, the photo-bioreactors may be spaced apart from one another to allow ambient light to reach the cultivation region for each photo-bioreactor. In some embodiments each photo-bioreactor is positioned over and spaced a distance from the underlying photo-bioreactor. To increase exposure to the ambient light the distance between the photo-bioreactors may be increased. Further, the photo-bioreactors may be placed at an angle as illustrated in FIG. 8. Preferably the photo-bioreactors are angled for maximum incident solar radiation for the particular geographic location. The photo-bioreactors may be angled from about 0 degrees (horizontal) to about 90 degrees (vertical). In some embodiments the photo-bioreactor may be angled from about 30 degrees to about 90 degrees. Further the position and angle of the photo-bioreactors may be changed during the day to track the sun or to maximize the incident solar radiation. Appropriate motors, hinges may be applied to the frame to change the position of the photo-bioreactor. Further the changing for the position of the photo-bioreactor may be automated such the photo-bioreactor is changed through out the day to maximize the incident solar radiation. In certain embodiments, the position of the photo-bioreactor may be automated to track with the position of the sun such that the incident solar radiation reaching the cultivation region in maximized.

More than one photo-bioreactor may be placed in series with one another to create a larger photo-bioreactor, or to provide different conditions for the organism being cultivated. For example, two or more photo-bioreactors may be in fluid communication with one another to effectively create a larger photo-bioreactor. The exit port of one photo-bioreactor may be connected in fluid communication with the inlet port of another photo-bioreactor. In some embodiments, different photo-bioreactors in the series may have different conditions, such as light exposure, temperature, nutrient insertion. In this way, the organism may be transferred from one photo-bioreactor to another and exposing the organism to different conditions. In further embodiments, the photo-bioreactors may be placed in parallel with one another.

While the photo-bioreactor has many uses, the use of the photo-bioreactor with respect to growing algae will be discussed. The photo-bioreactor may be placed outdoors in ambient conditions. The cultivation region is charged through the inlet port with the water and the desired strain of algae. Mono (unicultural) or multi strain algae mixtures may be utilized. Gases, including, but not limited to, carbon dioxide may be introduced to the photo-bioreactor through additional ports. In some embodiments a gas mixture containing about 5% carbon dioxide may be used. Further, nutrients may be introduced to the photo-bioreactor through the inlet port or through an additional port. The photo-bioreactor may be charged with the algae and water and left in a generally static environment in which the water and algae is not continuously circulating although the introduction of a gas containing carbon dioxide will bubble through the water and the cultivation region agitating the water to some degree. Alternatively, the water and algae may be continuously circulated through the bioreactor by pumping the water and algae into the photo-bioreactor through the inlet port. The water and algae will fill and flow through the cultivation region. The water and algae will exit the photo-bioreactor through the exit port where the water and algae may be circulated back to the same photo-bioreactor, sent to another photo-bioreactor, or to a station for separating the algae from the water, such as a centrifuge.

While various embodiments of the invention have been described above, the invention is limited only by the appended claims.

What is claimed is:

1. A photo-bioreactor comprising a cultivation region defined between two opposing layers of polymer film, wherein at least one of two opposing layers of polymer film absorbs less that about 15% of the natural sunlight, and an energy modulation region positioned adjacent to the cultivation region, wherein the energy modulation region comprises an energy modulating material comprising a phase change material.

2. The photo-bioreactor of claim 1 wherein the phase change material comprises a parrifin wax.

3. The photo-bioreactor of claim 2 wherein the paraffin wax has the general formula CₙH₂ₙ₊₂, where n may range from about 12 to about 20.

4. The photo-bioreactor of claim 1 wherein the phase change material is selected from the group consisting of tetradecane, hexadecane, octadecane, eicosane, Kenwax 18, Kenwax 19, 60% neopentyl glycol/40% pentaglycerine.

5. The photo-bioreactor of claim 1 wherein the phase change material comprises at least two different phase change materials.

6. The photo-bioreactor of claim 1 further comprising an insulation region positioned adjacent to the side of the cultivation region opposite the energy modulation region.
